(19) United States
(12) Patent Application Publication
(10) Pub. No.: US 2014/0298566 A1
(43) Pub. Date: Oct. 9, 2014

(54) Therapeutic Neck Pad

(71) Applicant: Renardo P. Rogers, Calumet City, IL (US)

(72) Inventor: Renardo P. Rogers, Calumet City, IL (US)

(21) Appl. No.: 14/244,022

(22) Filed: Apr. 3, 2014

Related U.S. Application Data
(60) Provisional application No. 61/810,146, filed on Apr. 9, 2013.

(51) Int. Cl. A61F 13/40

(52) U.S. Cl. CPC A61M 35/006
USPC 2/171.2

(57) ABSTRACT

Described is a therapeutic neck pad having an elongated strap with a widened central portion. The widened central portion can be placed on the front of a user's neck and covers a substantial portion thereof. The widened central portion includes an absorbent pad on which ointments, creams, lotions, or any desired topical medication can be placed. The absorbent pad brings the ointment into contact with a user's neck and provides a soft and comfortable surface for the user. The ends of the elongated strap have fasteners thereon. The neck pad can be wrapped around a user's neck and the ends of the elongated strap can be overlapped and secured together by means of the fasteners on each end.

### CROSS REFERENCE TO RELATED APPLICATION

This application claims the benefit of U.S. Provisional Application No. 61/810,146 filed on Apr. 9, 2013, entitled “Sore Throat Therapy Collar.” The above identified patent application is herein incorporated by reference in its entirety to provide continuity of disclosure.

### BACKGROUND OF THE INVENTION

### Field of the Invention

The present invention relates to therapeutic neck pads. More specifically, the present invention describes a neck pad comprising an elongated strap having a widened central portion on which an absorbent pad is disposed. The absorbent pad is adapted to receive ointment, creams, or other topical medication thereon. The pad is soft and flexible so that it can comfortably rest against the front of a user's neck. The ends of the strap comprise fasteners thereon for adjustably securing the therapeutic neck pad around a user's neck.

People commonly suffer from neck pain as result of a sore throat caused by various illnesses or because of muscle pain or tension. People may have a sore throat from having a viral illness such as a cold, laryngitis, mononucleosis, or influenza. Alternatively, a person may suffer a sore throat from a bacterial infection such as strep throat or tonsillitis, among others. Sore throats are particularly common in winter months and during periods of cold weather. Sore throats can be made worse by allergies or throat irritation resulting from yelling, smoking, or nasal drainage.

Various treatments are available for neck pain and sore throats, and the particular treatment used depends upon the type of neck injury or ailment. Treatments include application of heat or cold, soft-collar traction, topical anesthetic creams, topical pain-relief patches, and muscle relaxants, among others. Many topical treatments are commonly available for users to apply to their throats in order to soothe their throat or to relieve pain. Users may apply creams, lotions, gels, or ointments directly to their neck or throat to relieve pain or discomfort in that area. However, applying creams or lotions directly on the user's skin may result in the cream or lotion contacting a user's clothing, creating a stain. Further, when an article of clothing rubs against the lotion on the user's neck, the clothing may remove the lotion from the user's neck, eliminating the benefit of the lotion. As a result, users may have to clean stained clothes and reapply lotion that has been rubbed off of the user through contact with the user's clothing.

The present invention provides a therapeutic neck pad comprising an elongated strap having a first end and a second end. The elongated strap comprises a widened central portion on which an absorbent pad is disposed. The absorbent pad is adapted to receive a topical treatment such as a lotion or ointment thereon. The topical treatment and absorbent pad are placed against the user's neck and the elongated strap is wrapped around the user's neck. The first and second ends of the elongated strap comprise fasteners thereon that can be secured to one another when the first and second ends are overlapped. Thus, the elongated strap can be removably secured around a user's neck.

### DESCRIPTION OF THE PRIOR ART

Devices have been disclosed in the prior art that relate to neck pads. These include devices that have been patented and published in patent application publications. These devices generally relate to protective neck pads. The following is a list of devices deemed most relevant to the present disclosure, which are herein described for the purposes of highlighting and differentiating the unique aspects of the present invention, and further highlighting the drawbacks existing in the prior art.

One such prior art device is U.S. Design Patent Number D353,205 to Canavan, which discloses a design for an elongated heating pad adapted to be secured around a user's neck. The design includes an elongated heating pad having attachment means on each end thereof. However, Canavan fails to disclose a therapeutic neck pad having an absorbent pad disposed on a central portion thereof that is adapted to receive lotions, creams or ointments thereon.

U.S. Pat. No. 4,308,010 to De Woskin discloses neck pad material comprising three layers that are heat-sealed together, wherein the middle layer is thicker than the outer layers in order to provide cushioning. The neck pad material is in the form of tape and can be cut to a desired length. Accordingly, the neck pad material further has areas of heat seal arranged transversely thereon so that the neck pad material can be cut to form pads of selected length. Thus, De Woskin does not disclose a therapeutic neck pad having fasteners at each end that can be secured around a user's neck by overlapping the fasteners on each end of the therapeutic neck pad.

U.S. Patent Application Publication No. 2004/0226081 to Mrowka et al. discloses a protective neck pad for hockey players. The neck pad is dual layered and has a hard outer shell and a foam-cushioned inner shell for positioning against the back of a user's neck. The neck pad further comprises fasteners for securing the neck pad to the back of a user's helmet. In this way, the neck pad disclosed by Mrowka et al. is adapted to protect the exposed portion of the back of a hockey player's neck. Thus, Mrowka does not disclose a therapeutic neck pad having an absorbent pad adapted to receive ointment thereon, and wherein the neck pad wraps around a user's neck.

U.S. Patent Application Publication No. 2010/0305669 to Sasaki discloses a neck pad that is adapted to allow a user to warm or cool his or her neck. The neck pad comprises a housing that can be secured around a wearer's neck. The warming or cooling element is disposed within the housing on the back of the user's neck. The housing tapers towards the opposing ends of the device so that the housing is widest in the middle and relatively thin near the ends. Thus, Sasaki discloses a neck pad having heating or cooling means on the back of a user's neck and that is secured around the front of a user's neck. Sasaki fails to disclose a therapeutic neck pad on which ointments can be applied and that is intended for application to the front of a user's neck in order to soothe a sore throat.

U.S. Patent Application Publication No. 2012/0261446 to Stegmeyer discloses a shoulder strap having a pad member and a strap. The shoulder strap is adapted for placement over a user's shoulder for carrying an item such as a camera bag. The shoulder strap is adapted to reduce the load placed on the user's shoulder when carrying an item. The pad member has a slot extending therethrough in which the strap can be threaded. The pad member further comprises a high-friction surface for placement on a user's shoulder to prevent the pad from slipping on the user's shoulder. Thus, Stegmeyer fails to disclose a therapeutic neck pad that can be wrapped around a user's neck and secured by means of fasteners on each end of the therapeutic neck pad.

These prior art devices have several known drawbacks. Several devices in the prior art relate to neck pads, but fail to disclose a neck pad having an absorbent pad on which ointments and topical treatments can be placed. Thus, the prior art devices are not suited for soothing or treating a sore throat by facilitating application of topical treatments. Other patents disclose devices for applying heat to the back of a user's neck or devices for protecting the back of a user's neck. These devices are not adapted for treating a sore throat, but are instead concerned with treating muscle tension or pain or preventing injury from occurring to the back portion of a user's neck.

In light of the devices disclosed in the prior art, it is submitted that the present invention substantially diverges in design elements from the prior art and consequently it is clear that there is a need in the art for an improvement to existing therapeutic neck pad devices. In this regard the instant invention substantially fulfills these needs.

### SUMMARY OF THE INVENTION

In view of the foregoing disadvantages inherent in the known types of neck pads now present in the prior art, the present invention provides a new therapeutic neck pad wherein the same can be utilized for providing convenience for the user when applying topical ointments or other treatments to the front of a user's neck.

It is therefore an object of the present invention to provide a new and improved therapeutic neck pad device that has all of the advantages of the prior art and none of the disadvantages.

It is another object of the present invention to provide a therapeutic neck pad that can be adjustably secured around a user's neck.

Another object of the present invention is to provide a therapeutic neck pad that has an absorbent pad on which various types of topical treatments can be applied.

Yet another object of the present invention is to provide a therapeutic neck pad that shields a user's neck from cold weather.

Another object of the present invention is to provide a therapeutic neck pad that may be readily fabricated from materials that permit relative economy and are commensurate with durability.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a view of the interior surface of the therapeutic neck pad of the present invention.

FIG. 2 shows a view of the therapeutic neck pad as oriented in a loop for securement around a user's neck.

### DETAILED DESCRIPTION OF THE INVENTION

Reference is made herein to the attached drawings. Like reference numerals are used throughout the drawings to depict like or similar elements of the therapeutic neck pad. For the purposes of presenting a brief and clear description of the present invention, the preferred embodiment will be discussed as used for treating a sore throat. The figures are intended for representative purposes only and should not be considered to be limiting in any respect.

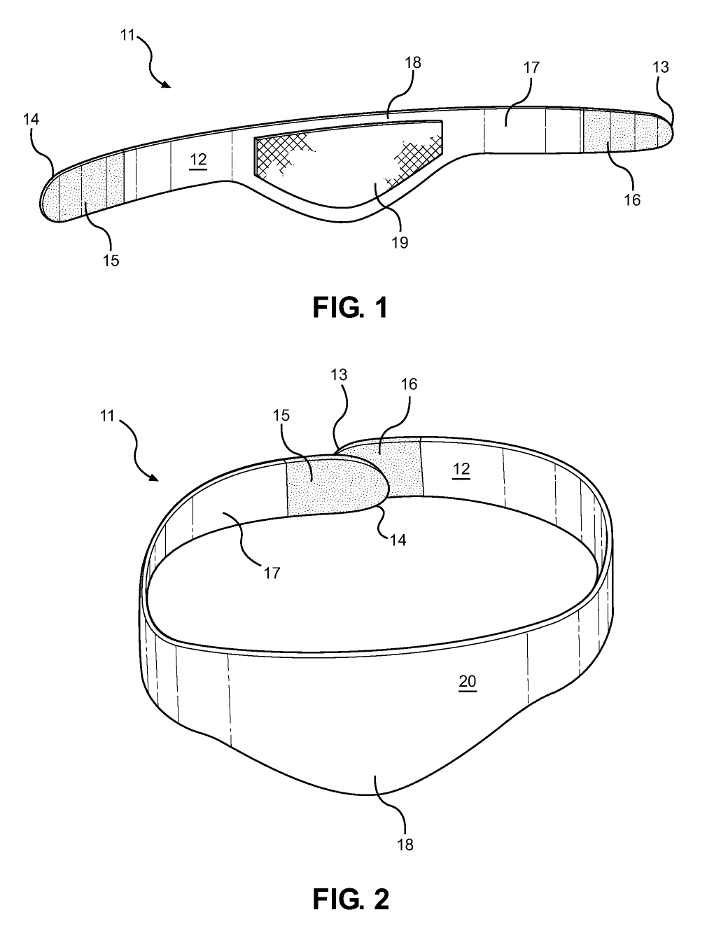

Referring now to FIG. 1, there is shown a view of the interior surface of the therapeutic neck pad of the present invention. The therapeutic neck pad 11 of the present invention comprises an elongated strap 17 having a first end 13 opposite a second end 14. A first fastener 16 is positioned on the first end 13 of the elongated strap 17, and a second fastener 15 is positioned on the second end 14 of the elongated strap 17. The elongated strap 17 further comprises an interior surface 12 to be positioned against a user's neck, and an exterior surface that faces outward from the user's neck. The elongated strap 17 comprises a flexible material such that the elongated strap 17 can be wrapped around a user's neck. The elongated strap 17 may be composed of a stretchable, breathable fabric material, such as nylon or rayon, or a breathable plastic. The elongated strap 17 can be comfortably positioned around a user's neck in order to shield the user's neck from cold weather and other elements. In some embodiments of the present invention, the interior surface 12 of the therapeutic neck pad, to be positioned against a user's neck, includes a soft lining thereon to provide added comfort to the user. The lining may be composed of cotton or other suitable soft and breathable materials. The lining is secured to the elongated strap by any suitable means such as by heat sealing or by sewing the lining onto the elongated strap.

The elongated strap 17 is substantially rectangular in shape and comprises a widened central portion 18. In some embodiments of the present invention, the widened central portion 18 comprises a rounded region that extends downward from a lower portion of the elongated strap 17 so as to provide greater coverage of the front of the user's neck. The widened central portion 18 extends downward towards the bottom of the user's neck. In other embodiments of the present invention, the widened central portion 18 may have a substantially rectangular shape. Further, the elongated strap 17 and the absorbent pad 19 may be scented to provide a pleasant aroma, such as lavender, jasmine, or chamomile for the user. Additionally, scents such as peppermint or menthol may be applied so as to serve as a nasal decongestant.

The widened central portion 18 further comprises an absorbent pad 19 positioned on the interior surface 12 thereof, and is adapted to be positioned against the front of a user's neck. The absorbent pad 19 is substantially the same shape as the widened central portion 18. The absorbent pad 19 is confined to the widened central portion 18 and does not otherwise extend along the elongated strap 17. The absorbent pad can comprise any suitable soft and absorbent material, including but not limited to gauze. The absorbent pad 19 is secured to the elongated strap 17 by any suitable fastening means such as heat sealing, adhesives, or sewing. The absorbent pad is adapted to receive creams, lotions, gels, ointments, and other topical treatments. Thus, the user can select a desired topical treatment, apply the treatment to the absorbent pad 19, and secure the present invention around the user's neck such that the absorbent pad 19 is in contact with the front of the user's neck.

In some embodiments of the present invention, the absorbent pad 19 is impregnated with a topical treatment. Thus, a user may simply apply the therapeutic neck pad 11 to the user's neck without having to first apply topical treatments. The impregnated treatment may include menthol, camphor, eucalyptus, benzocaine, or any other pain-relieving, warming or cooling substances. Such treatments may help to alleviate a sore throat, coughing, nasal congestion, and other symptoms of a common cold. The topical ointments may have any of a variety of effects. For example, the topical treatment may have a pain-relieving effect, a muscle relaxing effect, or a warming or cooling effect.

Referring now to FIG. 2, there is shown a perspective view of the therapeutic neck pad as oriented in a loop for securement around a user's neck. In operation, a topical treatment is applied to the absorbent pad and the absorbent pad is brought into contact with the front of a user's neck. In this way, the topical treatment is held against a user's throat so that it can be absorbed through the skin, and the treatment is prevented from being rubbed off by contact with the user's clothes or hands. With the widened central portion and the absorbent pad thereon disposed on the front of a user's neck, the first and second ends 13,14 of the elongated strap 17 are wrapped around the user's neck so that the therapeutic neck pad 11 forms a loop or a ring-like shape. The first end 13 of the elongated strap 17 is overlapped with the second end 14 of the elongated strap 17, or vice versa, and the fasteners 15,16 are used to secure the first and second ends 13,14 together. The fasteners 15,16 are secured in an area corresponding to the back of the user's neck. Any suitable fastener may be used including buttons, snaps, ties, hooks, clasps, or in a preferred embodiment of the present invention, hook and loop fastening material. Further, the elongated strap 17 is adjustably secured around a user's neck so that the therapeutic neck pad can fit users of various sizes and ages, and can be tightened or loosened as desired by the user.

In one embodiment of the present invention, a first end 13 has a fastener 16 on the interior surface 12 and the second end 14 has a fastener 15 on the exterior surface 20 of the therapeutic neck pad 11. Thus, the interior surface 12 of the second end 14 can be placed against a user's neck and does not have a fastener thereon, increasing comfort to the user. However, in this embodiment, the therapeutic neck pad 11 can only be secured around a user's neck by overlapping the first and second ends 13,14 in a particular order.

The present invention provides a therapeutic neck pad comprising an elongated strap having a widened central portion on which an absorbent pad is disposed. The ends of the elongated strap have fasteners thereon such that the elongated strap can be wrapped around a user's neck, and secured on a back portion of a user's neck by overlapping the ends of the elongated strap and securing the fasteners thereon. A user can apply various topical treatments to the absorbent pad for application to the front of the user's neck for the purpose of treating a sore throat or other neck ailment.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A therapeutic neck pad, comprising:
an elongated strap comprising an interior surface, an exterior surface, a first end, a second end, and a widened central portion;
a pad disposed on said interior surface of said widened central portion adapted to receive ointment thereon for positioning on a front portion of a user's neck;
one or more fasteners disposed on said first end and on said second end of said elongated strap;
wherein said elongated strap is adapted to be removably secured around a user's neck by overlapping and securing said fasteners on said first end to said fasteners on said second end.

2. The neck pad of claim 1, wherein said pad is absorbent.

3. The neck pad of claim 1, wherein said pad comprises gauze.

4. The neck pad of claim 1, wherein said elongated strap is flexible.

5. The neck pad of claim 1, wherein said elongated strap comprises a stretchable fabric material.

6. The neck pad of claim 1, wherein said one or more fasteners comprise hook and loop fastening material.

7. The neck pad of claim 1, wherein said elongated strap is substantially rectangular.

8. The neck pad of claim 1, wherein said widened central portion comprises a curved projection extending from a bottom portion of said elongated strap.

9. The neck pad of claim 1, wherein said pad is impregnated with a topical ointment.

10. The neck pad of claim 1, wherein said pad is shaped similarly to said widened central portion of said elongated strap.

11. The neck pad of claim 1, wherein said elongated strap is scented.
